(19) United States
(12) Patent Application Publication
(10) Pub. No.: US 2008/0272052 A1
(43) Pub. Date: Nov. 6, 2008

(54) Method for Producing and Dewatering Cyclic Formals

(75) Inventors: Reinhard Wagener, Hofheim (DE); Michael Haubs, Bad Kreuznach (DE); Juergen Lingnau, Mainz-Laubenheim (DE); Matthias Goring, Hofheim (DE); Michael Hoffmockel, Niedernhausen (DE)

Correspondence Address: CONNOLLY BOVE LODGE & HUTZ, LLP, P O BOX 2207, WILMINGTON, DE, 19899, US

(73) Assignee: TICONA GmbH, Kelsterbach (DE)

(21) Appl. No.: 11/665,028

(22) PCT Filed: Oct. 6, 2005
(86) PCT No.: PCT/EP05/10760

§ 371 Date: Feb. 7, 2008

(30) Foreign Application Priority Data
Oct. 8, 2004 (DE) 10 2004 049 055.4

(51) Int. Cl. B01D 61/36; C07D 317/12

(52) U.S. Cl. 210/637; 210/638; 210/640

(57) ABSTRACT

Processes are described which comprise: (a) providing a mixture comprising a cyclic formal and water, wherein the mixture has a cyclic formal concentration and a water concentration; (b) bringing the mixture into contact with an organically selective membrane; (c) creating a pressure differential across the membrane; and (d) obtaining a permeate having a lower water concentration and a higher cyclic formal concentration than the mixture, and a retentate having a higher water concentration and a lower cyclic formal concentration than the mixture.

The invention relates to a process for preparing anhydrous cyclic formals.

Cyclic formals can be prepared by acid-catalyzed reaction of dihydric alcohols (dialcohols) and formaldehyde. The industrially most important cyclic formal is 1,3-dioxolane (dioxolane). It is prepared industrially by acid-catalyzed reaction of aqueous formaldehyde with ethylene glycol. Dioxolane can be removed from the reaction mixture by distillation, but is always accompanied by water because the two components form an azeotrope with approx. 93% by weight of dioxolane. For the solution of this separation problem, numerous processes have been proposed, most of which utilize extraction or extractive rectification in order to overcome the azeotropic point of the water/dioxolane mixture.

U.S. Pat. No. 5,690,793 and U.S. Pat. No. 5,695,615 disclose processes for purifying cyclic formals in which water is removed in an extractive distillation with polar nonvolatile solvents.

U.S. Pat. No. 5,456,805 describes the separation of dioxolane and water from the reaction of formaldehyde with ethylene glycol by extractive distillation with n-pentane.

DE 1 279 025 teaches the separation of dioxolane and water from the reaction of formaldehyde with ethylene glycol by extractive distillation with alkaline aqueous solutions.

BE 669 480 discloses a process for extraction of dioxolane from aqueous mixtures with chlorinated hydrocarbons and subsequent alkaline scrubbing of the crude dioxolane.

JP 07 285958 teaches a process in which the azeotrope of water and dioxolane is extracted with hydrocarbons in the liquid phase and then the organic phase is distilled to give the pure dioxolane.

DE 39 39 867 A1 describes the removal of water from neutral organic solvents with the aid of a composite membrane comprising a separating layer composed of crosslinked polyvinyl alcohol. This document does not give any indication to a corresponding use in the case of cyclic formals, nor is there any disclosure regarding the use of organically selective membranes.

The prior art processes are in need of improvement because, as well as water and cyclic formals, they introduce a third substance into the process as an extractant or azeotroping agent. This third substance normally has to be purified in a separate cycle in order to be able to be used again. If this does not succeed completely, partial disposal of the third substance leads to complicated subsequent purification or to pollution of the environment. In any case, the additional separating operations require additional energy for their operation.

There is therefore a need for a process for preparing anhydrous cyclic formals
which does not require a third substance as an extractant or azeotroping agent;
which does not present any disposal problems in the case of the incomplete recovery of the third substance;
and which works with reduced energy consumption.

It has been found that, surprisingly, pervaporation or vapor permeation of cyclic formals, especially of 1,3-dioxolane, and water with suitable organically selective membranes affords very good separation factors and high permeate flows. The membrane separation of cyclic formals from water can also be operated at elevated temperatures with further enhanced permeate flows.

The invention therefore provides a process for removing cyclic formals, especially dioxolane, from mixtures with water, which comprises
a) contacting the mixture comprising cyclic formal and other substances with an organically selective membrane,
b) applying a pressure difference over the membrane and
c) obtaining, on the permeate side of the membrane, a product which has a higher concentration of cyclic formal and a lower concentration of water than the starting mixture.

The invention further provides a process for purifying cyclic formals, which comprises
a) feeding a liquid mixture comprising at least one cyclic formal and water to an organically selective pervaporation membrane,
b) obtaining from the pervaporation a liquid retentate with a content of water and a vaporous permeate with a higher content of cyclic formal,
c) purifying the vapor stream from step b) by distillation, extractive distillation, crystallization, extraction or a further membrane separation to give anhydrous cyclic formal of the desired quality.

In another embodiment of the invention, the membrane separation is not performed as a pervaporation with a liquid feed but rather as a vapor permeation with a vaporous starting mixture of the cyclic formal with water.

The invention therefore further provides a process for purifying cyclic formals, which comprises
a) feeding a vaporous mixture comprising at least one cyclic formal and water to an organically selective vapor permeation membrane,
b) obtaining from the vapor permeation a vaporous retentate with a higher content of water and a vaporous permeate with a higher content of cyclic formal,
c) purifying the vapor stream from step b) by distillation, extractive distillation, crystallization, extraction or a further membrane separation to give anhydrous cyclic formal of the desired quality.

Cyclic formals are obtained in a cyclization reaction from dialcohols and formaldehyde. Typical representatives are 1,3-dioxolane (from ethylene glycol), 1,3-dioxepane (from 1,4-butanediol), diethylene glycol formal, 4-methyl-1,3-dioxolane (from 1,2-propanediol), 1,3-dioxane (from 1,3-propanediol), 4-methyl-1,3-dioxane (from 1,3-butanediol) and 1,3,5-trioxepane (from ethylene glycol and two molecules of formaldehyde). Preference is given to 1,3-dioxolane.

Suitable catalytically active acids are, for example, mineral acids such as sulfuric acid, phosphoric acid, or aliphatic or aromatic sulfonic acids such as methanesulfonic acid, trifluoromethanesulfonic acid, benzenesulfonic acid, toluenesulfonic acid, naphthalenesulfonic acid, or else highly acidic ion exchange resins or heteropolyacids such as polyphosphoric acid, tungstophosphoric acid or molybdophosphoric acid.

The reaction can be conducted according to the prior art in a stirred tank reactor with attached distillation column or as a reactive distillation column. The mixture of cyclic formal and water obtained at the top of this column already contains more than 30% by weight, preferably more than 40% by weight and more preferably more than 50% by weight of cyclic formal. In addition to the cyclic formal and water, the mixture may also comprise other constituents of the reaction mixture, such as dialcohol or formaldehyde, in small concentrations.

In a preferred embodiment of the invention, the feed mixture consisting essentially of cyclic formal and water is obtained as a distillate or exhaust vapor from the reaction of a dialcohol with formaldehyde under acidic catalysis.

The invention therefore further provides a process for preparing cyclic formals from dialcohols and formaldehyde, which comprises
a) reacting the dialcohol and the formaldehyde with catalysis by a suitable acid,
b) decompressing a vaporous mixture essentially comprising the cyclic formal and water out of the reaction vessel,
c) condensing the vaporous mixture obtained in step b),
d) feeding the condensed mixture from step c) to an organically selective pervaporation membrane,
e) obtaining from the pervaporation a liquid retentate with higher water content and a vaporous permeate with higher content of cyclic formal,
f) purifying the vapor stream from step e) by distillation, extractive distillation, crystallization, extraction or a further membrane separation to give cyclic formal of the desired quality.

In a particularly preferred embodiment of the invention, the vaporous mixture from step b) is not condensed, but rather fed as vapor to an organically selective vapor permeation membrane. This procedure is particularly advantageous with regard to the evaporation energy to be applied, because it utilizes the energy content of the exhaust vapor from the reaction vessel.

The invention therefore further provides a process for preparing cyclic formals from dialcohols and formaldehyde, which comprises
a) reacting the dialcohol and the formaldehyde with catalysis by a suitable acid,
b) decompressing a vaporous mixture essentially comprising the cyclic formal and water out of the reaction vessel,
c) keeping the mixture obtained in step b) in vaporous form and optionally heat-treating it,
d) feeding the vaporous mixture from step c) to an organically selective vapor permeation membrane,
e) obtaining from the vapor permeation a vaporous retentate with higher water content and a vaporous permeate with higher content of cyclic formal,
f) purifying the vapor stream from step e) by distillation, extractive distillation, crystallization or a further membrane separation to give cyclic formal of the desired quality.

For the process according to the invention, membranes which allow organic components, especially cyclic formals, to permeate preferentially over water are used. Suitable membranes for the process according to the invention may be used equally in the pervaporation procedure with liquid membrane feed and in the vapor permeation procedure. The separation-active layer of the membrane consists generally of crosslinked polymers which are rubber-like (under the separating conditions). In a preferred embodiment, the rubber-like polymers consist of polydimethylsiloxane or modified polydimethylsiloxanes such as polyoctylmethylsiloxane or another polyalkylmethyl- or polyarylmethylsiloxane. Likewise suitable are rubber-like polyphosphazenes. In a preferred embodiment of the invention, the membrane is subjected to subsequent crosslinking, for example radiative crosslinking, in order to increase its selectivity and solvent resistance.

The separation-active layer of the membrane has a thickness of 1-200 μm, preferably 2-50 μm and more preferably 4-10 μm.

The separation factor α of the membrane process depends upon the selectivity of the membrane and the pressure ratio over the membrane. The separation factor α of the membrane process can be determined experimentally as follows:

α=(yₚ/xₚ)/(y_{f}/x_{f})
where: yₚ=proportion by mass of the cyclic formal in the permeate
   xₚ=proportion by mass of the water in the permeate
   y_{f}=proportion by mass of the cyclic formal in the feed
   x_{f}=proportion by mass of the water in the feed
      and is, for the separation of the cyclic formal from water, generally α>5, preferably α>10 and more preferably α>15.

To perform the inventive removal of dioxolane and/or other cyclic formals, a pressure difference is applied over the membrane. This is typically done by applying a reduced pressure on the permeate side of the membrane. However, the pressure difference can also be increased by increasing the partial pressure of the dioxolane on the feed side of the membrane.

The permeation rate of the membrane, measured at atmospheric pressure and 40° C. on the feed side and a permeate pressure of 10 mbar, is above 1 kg/m²/h, preferably above 3 kg/m²/h and more preferably above 5 kg/m²/h. Under suitable operating conditions, for example at operating temperatures of above 50° C., the permeation rate of the membrane in the separation process according to the invention can achieve values of above 10 kg/m²/h or even above 15 kg/m²/h.

A particular advantage of the process is that good separating performances are achieved even with a heated feed. It is known to those skilled in the art that organically selective pervaporation membranes are swollen at elevated temperatures by polar aprotic solvents such as the cyclic formals and can loose their selectivity. In the process according to the invention, separation factors of α>5, preferably α>10, are still achieved even at feed temperatures of T≧40° C. In a preferred embodiment of the invention, the feed to the pervaporation or vapor permeation membrane is adjusted to a temperature of T>40° C.

In a preferred embodiment of the invention, the permeate has a composition whose content of cyclic formal is above the binary azeotrope of the formal with water. In the case of the preferred dioxolane, the permeate contains preferably over 93% by weight and more preferably over 95% by weight of dioxolane. The still water-contaminated cyclic formal thus obtained can be worked up to the desired purity in further separation steps. Suitable measures for this purpose are, for example, distillation, extractive distillation, crystallization, extraction or a further membrane separation.

Further preferred embodiments of the invention are evident from the subclaims.

### EXAMPLE 1

A mixture of 50% by weight of dioxolane and 50% by weight of water which has been adjusted to a temperature of 40° C. is fed in pumped circulation to a pervaporation test cell. The test cell is equipped with a polydimethylsiloxane composite membrane on a porous polyacrylonitrile support membrane. The thickness of the separation-active siloxane layer is 8 μm. In the permeate space, a pressure of 10 mbar absolute is established. The permeate is condensed in a cold trap at 0-5° C. Once steady-state conditions have been established, the cold trap is changed and an analysis of the permeate which is then obtained is performed. 94.7% by weight of dioxolane is obtained, corresponding to a separation factor of α=18 in the permeate at a permeation rate of 10 kg/m²/h.

### EXAMPLE 2

Further experiments in the test cell are performed analogously to Example 1 with the following results. A membrane with a separation-active layer composed of polyoctylmethylsiloxane on a porous polyacrylonitrile support membrane is used:

TABLE 1

| | | Example No. | | | |
|---|---|---|---|---|---|
| | | 2.1 | 2.2 | 2.3 | 2.4 |
| Dioxolane content | [% by | 60 | 60 | 20 | 20 |
| Feed | wt.] | | | | |
| Feed temperature | [° C.] | 30 | 40 | 40 | 80 |
| Membrane | [μm] | 4 | 4 | 4 | 4 |
| thickness | | | | | |
| Dioxolane content | [% by | 96.6 | 96.0 | 86.3 | 81.8 |
| Permeate | wt.] | | | | |
| Separation factor | [1] | 19 | 16 | 25.2 | 18 |
| Permeation rate | [kg/m²/h] | 8.0 | 11.5 | 5.2 | 40.0 |

### EXAMPLE 3

100 liters/h of a mixture of dioxolane and water are fed to a pervaporation pilot apparatus. The pilot apparatus is equipped with 1 m² of a polydimethylsiloxane membrane on a porous polyacrylonitrile support membrane. The membrane was subsequently radiation-crosslinked. The permeate is condensed at approx. 0° C. and collected in a cooled vessel. Once steady-state conditions have been established, analyses of the feed and of the permeate are performed. Table 2 summarizes the significant operating conditions and results:

TABLE 2

| | | Example No. | | | |
|---|---|---|---|---|---|
| | | 3.1 | 3.2 | 3.3 | 3.4 |
| Dioxolane content | [% by | 59.4 | 58.8 | 57.0 | 56.7 |
| Feed | wt.] | | | | |
| Feed temperature | [° C.] | 20 | 14 | 44 | 44 |
| Pressure in the | [mbar a] | 90 | 95 | 95 | 100 |
| permeate space | | | | | |
| Dioxolane content | [% by | 95.2 | 95.5 | 93.1 | 93.2 |
| Permeate | wt.] | | | | |
| Separation factor | [1] | 14.4 | 15.6 | 10.7 | 10.5 |
| Permeation rate | [kg/m²/h] | 4.0 | 4.0 | 10.0 | 8.0 |

1-25. (canceled)

26. A process comprising:
(a) providing a mixture comprising a cyclic formal and water, wherein the mixture has a cyclic formal concentration and a water concentration;
(b) bringing the mixture into contact with an organically selective membrane;
(c) creating a pressure differential across the membrane; and
(d) obtaining a permeate having a lower water concentration and a higher cyclic formal concentration than the mixture, and a retentate having a higher water concentration and a lower cyclic formal concentration than the mixture.

27. The process according to claim 26, further comprising subjecting the permeate to one or more purifications selected from the group consisting of distillation, extractive distillation, crystallization, extraction, a further membrane separation, and combinations thereof.

28. The process according to claim 26, wherein the mixture is provided as a liquid, and wherein the membrane comprises a pervaporation membrane.

29. The process according to claim 26, wherein the mixture is provided as a vapor, and wherein the membrane comprises a vapor permeation membrane.

30. The process according to claim 27, wherein the mixture is provided as a liquid, and wherein the membrane comprises a pervaporation membrane.

31. The process according to claim 27, wherein the mixture is provided as a vapor, and wherein the membrane comprises a vapor permeation membrane.

32. The process according to claim 26, wherein the mixture comprises a product obtained by reacting a dialcohol and formaldehyde in the presence of a suitable acidic catalyst.

33. The process according to claim 32, wherein the suitable acidic catalyst comprises one or more materials selected from the group consisting of sulfuric acid, phosphoric acid, aliphatic sulfonic acids, aromatic sulfonic acids, strongly acidic ion exchange resins, heteropolyacids, and mixtures thereof.

34. The process according to claim 28, wherein the mixture comprises a liquid product obtained by reacting a dialcohol and formaldehyde in the presence of a suitable acidic catalyst to form an initial product, and condensing the initial product.

35. The process according to claim 26, wherein the cyclic formal comprises at least one selected from the group consisting of 1,3-dioxolane, 1,3-dioxepane, diethylene glycol formal, 4-methyl-1,3-dioxolane, 1,3-dioxane, 4-methyl-1,3-dioxane, 1,3,5-trioxepane, and mixtures thereof.

36. The process according to claim 26, wherein the cyclic formal comprises 1,3-dioxolane.

37. The process according to claim 26, wherein the cyclic formal concentration of the mixture is greater than 30% by weight.

38. The process according to claim 32, wherein the cyclic formal concentration of the mixture is greater than 30% by weight.

39. The process according to claim 26, wherein the membrane comprises a material selected from the group consisting of polyalkylmethylsiloxanes, polyarylmethylsiloxanes, polyphosphazenes, and mixtures thereof.

40. The process according to claim 26, wherein the membrane comprises a polysiloxane which has been subjected to radiative crosslinking.

41. The process according to claim 26, wherein the membrane comprises a separation-active layer having a thickness of 1-200 μm.

42. The process according to claim 41, wherein the separation-active layer comprises a material selected from the group consisting of polyalkylmethylsiloxanes, polyarylmethylsiloxanes, polyphosphazenes, and mixtures thereof.

43. The process according to claim 26, wherein the process has a separation factor α greater than 5.

44. The process according to claim 26, wherein the mixture is brought into contact with the membrane at a temperature greater than 40° C.

45. The process according to claim 26, wherein the cyclic formal concentration of the permeate is greater than 93% by weight.
